(19) United States
(12) Patent Application Publication
(10) Pub. No.: US 2006/0239946 A1
(43) Pub. Date: Oct. 26, 2006

(54) Cosmetic composition and method for treating keratinous materials, comprising a photodimerizable compound

(76) Inventors: Henri Samain, Bievres (FR); Gabin Vic, Venette (FR); Aude Livoreil, Paris (FR); Franck Giroud, Cllchy (FR)

Correspondence Address: C. IRVIN MCCLELLAND;OBLON, SPIVAK, MCCLELLAND, MAIER & NEUSTADT, P.C., 1940 DUKE STREET, ALEXANDRIA, VA, 22314, US

(21) Appl. No.: 10/538,358

(22) PCT Filed: Dec. 12, 2003
(86) PCT No.: PCT/FR03/03698

§ 371 Date: Apr. 13, 2006

(30) Foreign Application Priority Data
Dec. 15, 2002 (FR) 0215860
Feb. 20, 2003 (US) 60448112

(51) Int. Cl. A61K 8/81; A61K 8/49

(52) U.S. Cl. 424/63; 424/70.16

(57) ABSTRACT

The invention concerns generally a cosmetic composition comprising at least one cosmetic active principle and at least one photodimerizable compound enabling a material deposit to be provided on the keratinous materials, which is resistant to washing, and whereof the location is precisely controlled, capable of providing long-lasting cosmetic properties to keratinous materials, and of being removed at any time.

The present invention relates generally to a cosmetic composition comprising at least one cosmetic active agent and at least one photodimerizable material for producing a deposit of material on keratin materials, and in particular on the hair, which is resistant to washing, the placement of which is controlled precisely, which can afford the keratin materials long-lasting cosmetic properties, and which can be removed at any time.

The resistance of a deposit of material to washing may be afforded by low solubility of the deposit in aqueous detergent media.

Bringing about crosslinking by the action of light (photo-crosslinking) makes it possible to selectively crosslink a deposit, in particular on specific areas of keratin materials that have been coated beforehand with the photo-crosslinking composition, and thus to create a long-lasting deposit on some areas and not on others. The non-irradiated parts of the deposit (for example those covered with a mask), which are thus non-crosslinked, remain soluble. They may then be readily removed by rinsing or shampooing after the irradiation. It thus becomes possible, for example, to create patterns on the keratin materials and images using various stencils and masks with a photo-crosslinking composition, for example a coloured composition, or alternatively to treat only particular sensitized areas, for instance the ends in the case of the hair, with a conditioning composition or the like.

It is known practice to those skilled in the art to use photo-crosslinking materials such as materials derived from vinyl monomers, and especially (meth)acrylate monomers.

Thus, patent U.S. Pat. No. 5,300,285 describes a process for waving the hair and a composition for carrying out this process, and especially a silicone-based neutralizing composition, comprising a silicone containing a vinyl function, a free-radical photoinitiator and a solvent. This composition acts as a substitute for neutralizing solutions based on hydrogen peroxide when it is applied to the hair after a process of permanently reshaping the hair and photo-crosslinking the composition. This composition has the advantage of functioning simultaneously as a keratin-crosslinking agent while at the same time affording the hair advantages such as conditioning and a soft feel. The photo-crosslinking of the composition is triggered by the photoinitiator (generally an acetophenone derivative), which releases a radical on irradiation in the UVA range (350-385 nm).

Compositions that are resistant to various types of attack, for instance water or mechanical impacts, and also compositions that afford gloss and good staying power, are also sought in other cosmetic fields, for instance nail varnishes.

Unfortunately, the photo-polymerization of silicone containing a vinyl function in the presence of a photoinitiator leads to an irreversible photo-crosslinking of the silicone containing a vinyl function. In other words, once photo-crosslinked, the deposit cannot be removed from the hair readily and at any time.

Furthermore, besides the toxicologically unfavourable aspects of these monomers and of the photoinitiator, compositions containing these materials derived from vinyl monomers are known to be unstable, in particular on account of their sensitivity to oxygen and to moisture. Moreover, the photo-crosslinking is occasionally obtained by means of prolonged irradiation, resulting in risks of degradation of the hair under the action of UV.

Finally, Japanese patent JP 09 249 812 describes interpenetrating networks containing chelate-forming molecules and their uses, and more particularly compositions containing polymers of partially saponified polyvinyl acetate type containing stilbazolium groups, crosslinked and containing chelating agents. This precrosslinked material may then be used in unmodified form in medical or cosmetic applications. However, no mention is made in the said patent (JP 09 249 812) of the application of a composition containing polymers that are not crosslinked on the hair or the skin before irradiation.

The object of the present invention is thus to provide a cosmetic composition, which, after application to keratin materials, in particular to keratin fibres such as the hair, is photo-crosslinked in an optionally reversible manner, and results in a crosslinked deposit which does not redissolve, which is resistant to washing and which does not degrade the keratin fibres, thus ensuring that the cosmetic effects afforded by this deposit are long-lasting.

For the purposes of the present patent application, the expression “reversible photo-crosslinking of a material or a composition” means crosslinking of the material or of the composition by the action of light, resulting in a deposit that may be removed from the hair easily and at any time. Thus, irradiation in the UVB range (around 250 nm) leads to reformation of the original double bonds and to the disappearance of the crosslinking.

In particular, one subject of the present invention is a cosmetic composition for treating keratin materials, comprising at least one cosmetic active agent, characterized in that it also comprises at least one compound comprising at least one photodimerizable group, this compound having a molecular mass of greater than 500 g/mol.

Advantageously, in the compositions of the invention, the compound comprising at least one photodimerizable group has a molecular mass of greater than 700 g/mol, more advantageously greater than 850 g/mol and even more preferably greater than 1 000 g/mol.

For the purposes of the present invention, the term “cosmetic active agent” means any active compound that has cosmetic or dermatological activity.

For the purposes of the present invention, the term “photodimerizable group” means a chemical group that leads to photodimerization reactions on irradiation.

For the purposes of the present invention, the term “photodimerization” means a chemical reaction between two double bonds (of 2+2 type) or two pairs of double bonds (of 4+4 type).

The case of a reaction between two double bonds may be represented schematically in the following manner:

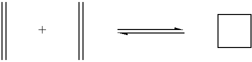

These photodimerization reactions are defined in the document “Advanced Organic Chemistry”, J. Marck, 4th edition, Wiley Interscience, NY, 1992, p. 855.

The materials containing photodimerizable groups according to the invention have the advantage of being stable with respect to oxygen, moisture and heat, and of leading to reversible crosslinking.

In addition, the photodimerizable groups according to the invention are very photosensitive. Consequently, an irradiation, even of low energy, leads to rapid and efficient crosslinking of the material, which, in the case of an application in cosmetics, induces an irradiation of short duration and of low energy, which does not result in degradation of the keratin materials. This is especially advantageous for application in haircare or in nail varnishes.

Preferably, the photodimerizable groups that may be used according to the invention are chosen from the monovalent radicals of formulae: 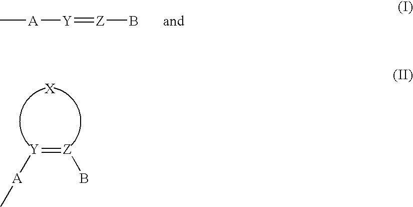
in which:
Y and Z denote, independently of each other, a carbon or nitrogen atom,
A is a divalent bonding group chosen from alkylene radicals, cycloaromatic radicals, heterocyclic radicals, carbonyl radicals and alkenylene radicals, and combinations thereof,
B is a monovalent group chosen from alkyl radicals, cycloaromatic radicals, heterocyclic radicals, carbonyl radicals and alkenyl radicals, and combinations thereof,
X is a divalent group chosen from alkylene radicals, cycloaromatic radicals, heterocyclic radicals, carbonyl radicals and alkenylene radicals, and combinations thereof, and
each of the groups mentioned optionally being substituted with one or more groups chosen from alkyl, hydroxyl, amino, monoalkylamino or dialkylamino, halogen, aryl, carboxyl, alkoxy, alkoxycarbonyl, hydrogenocarbonyl, sulphonato, amide and acyl groups.

The dimerizable groups according to the invention are especially those mentioned in patents U.S. Pat. No. 2,811,510, EP 0 313 220, EP 0 313 221, EP 092 901, GB 2 030 575 and GB 2 076 826, and also in the articles “Chemical Review”, vol. 83.5 1983, p. 507, “Polym. Paint Colour Journal” 1988, 178, p. 209 and “Current Trends in Polymer Photochemistry”, Ellis Morwood edition, NY, 1995.

Examples that may be mentioned more particularly are photodimerizable groups chosen from the monovalent radicals of the following compounds:
stilbene,
styrylpyridinium (stilbazolium) of formula: 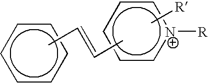
   in which
R represents a hydrogen atom or an alkyl or hydroxyalkyl group, and
R′ represents a hydrogen atom or an alkyl group,
styrylazolium of formula: 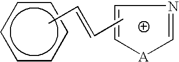
   in which A denotes a sulphur atom, an oxygen atom or a group NR′ or C(R′)₂, R and R′ being as defined above,
styrylpyrazine,
chalcone,
(thio)cinnamate and (thio)cinnamamide,
maleimide,
(thio)coumarin,
thymine,
uracil,
butadiene,
anthracene,
pyridone,
pyrrolizinone,
acridizinium salts,
furanone,
phenylbenzoxazole, and
derivatives thereof.

Such chemical groups contain activated double bonds, and as such the photodimerization of these double bonds is triggered spontaneously in the UVA range, without a photoinitiator being necessary.

Advantageously, in the compositions of the invention, the compound comprising at least one photodimerizable group is a polymer. Even more advantageously, it is in the form of particles, in particular of dispersed particles.

For the purposes of the present invention, the term “photoinitiator” means a compound that initiates the photodimerization reaction and releases a radical on irradiation, especially in the UV range.

Consequently, when the composition according to the invention comprises a compound comprising at least one photodimerizable group chosen from the photodimerizable groups mentioned above, it is preferably free of photoinitiator.

The compounds containing photodimerizable group(s) according to the invention are preferably not colorants.

The photodimerizable groups according to the invention may be readily grafted onto any type of support compound.

Advantageously, the support compound is a particle-forming filler.

The term “particles” includes mineral particles and organic particles. The mineral particles may consist of silicon, calcium, magnesium, zinc, aluminium, titanium, zirconium or cerium oxides, oxide dihydrates, hydroxides, carbonates, sulphides, silicates or phosphates, nacres, micas, particles consisting of native metals, i.e. alkali metals, alkaline-earth metals, transition metals or rare-earth metals, which may or may not be coated, and alloys thereof. Among the metals that are preferred are aluminium, copper, silver, gold, indium, iron, platinum, nickel, molybdenum, silicon, titanium, tungsten, antimony, palladium, cadmium, zinc, tin, and selenium, and alloys thereof. Among the above list, gold, silver, palladium and platinum, and alloys thereof, are preferred, silver and gold being the preferred metals.

The organic particles may consist of latices, polystyrene, polystyrene derivatives, silicones, fluoro polymers, polypropylene, polyethylene, poly(meth)-acrylic acid, polymethacrylate, polyurethane, polyamide, polycarbonate, polyvinyl chloride, polyvinyl acetate, fluoropolymers, polyethylenes, polypropylene, polyisobutylene, poly(1-butylene), copolymers and blends of the polymers mentioned, alkyl cellulose, hydroxyalkyl cellulose, cellulose ethers, cellulose esters, hydroxypropylcellulose, hydropropyldextran, hydroxypropylmethyl cellulose, cellulose acetate, carboxyethylcellulose, cellulose sulphate, dextran sulphate, polyvinyl alcohol, polyethylene oxide, polyvinyl chloride or polyvinylpyrrolidone.

The polystyrene particles are commercially available from Polyscience Inc. (Warrington, Pa.) or Duke Scientific Corporation (Palo Alto, Calif.).

These organic particles may be crosslinked using crosslinking agents, for instance divinylbenzene, glutaraldehyde, 1,4-bis(acryloyl)piperazine, carbodiimides, N-hydroxysuccinimide and derivatives, divinyl sulphone, dithiobis(succinimidyl) propionate or N-succinimidyl-3-(2-pyridyldithio)propionate.

Preferably, the support compound is chosen from polymers and even more preferably from poly(vinyl) polymers and polydiorganosiloxanes.

Among the poly(vinyl) polymers that are particularly preferred are poly(vinyl acetates), preferably partially saponified.

The compound(s) comprising at least one photodimerizable group according to the invention are thus, advantageously, a polymer that is soluble or dispersed in the composition, or, even more advantageously, a polymer that is adsorbed onto polymer particles, which are themselves dispersed in the composition. In the latter case, the polymer particles are very preferably vinyl polymer particles and better still poly(vinyl acetate) particles.

The invention is most particularly directed, as compound(s) comprising at least one photodimerizable group, towards aqueous dispersions of a partially saponified polymer of polyvinyl acetate type bearing stilbazolium groups, placed in contact, especially blended, with polyvinyl acetate particles.

The photodimerizable material may also form all or part of a material of core/shell type as defined in patent application FR 2 838 052, filed by the Applicant.

The compound(s) comprising at least one photodimerizable group according to the invention preferably represent(s) 0.01% to 25%, better still 0.1% to 20% and even better still 1% to 15% of the total weight of the composition.

The cosmetic composition according to the invention may also comprise an effective amount of at least one photosensitizer.

For the purposes of the present invention, the term “photosensitizer” means an ingredient that modifies the irradiation wavelength, thus triggering the photodimerization reaction.

For example, the photodimerization of dimethylmaleimide groups is triggered by an irradiation centred on the wavelength range from 270 to 300 nm. In the presence of a photosensitizer such as thioxanthone, the photodimerization becomes effective with an irradiation centred on the wavelength range from 360 to 430 nm.

Among the photosensitizers that may be used according to the invention, mention may be made especially of thioxanthone, rose bengal, phloxin, eosin, erythrosin, fluorescein, acriflavine, thionine, riboflavine, proflavine, chlorophylls, haematoporphyrin and methylene blue, and mixtures thereof.

In practice, the photosensitizer that may be used according to the invention represents 0.00001% to 5% of the total weight of the composition.

The cosmetic active agent(s) according to the invention is (are) chosen from:
hydrolysed or non-hydrolysed, modified or unmodified saccharides, oligosaccharides and polysaccharides,
amino acids, oligopeptides, peptides, hydrolysed or non-hydrolysed, modified or unmodified proteins, polyamino acids and enzymes,
branched or unbranched fatty alcohols and fatty acids,
animal, plant or mineral waxes,
ceramides and pseudoceramides,
hydroxylated organic acids,
UV-screening agents,
antioxidants and free-radical scavengers,
chelating agents,
antidandruff agents,
seborrhoea regulators,
calmants,
cationic surfactants,
cationic or amphoteric polymers,
organomodified or non-organomodified silicones,
mineral, plant or animal oils,
polyisobutenes and poly(α-olefins),
esters,
soluble or dispersed anionic polymers,
soluble or dispersed nonionic polymers,
reducing agents,
dyestuffs, and especially hair dyes,
foaming agents,
film-forming agents,
particles,
and mixtures thereof.

In general, the compounds of modified or unmodified, optionally hydrolysed saccharide, oligosaccharide and polysaccharide type, which may be used in the present invention, are chosen from those described especially in “Encyclopedia of Chemical Technology”, Kirk-Othmer, Third Edition, 1982, volume 3, pp. 896-900 and volume 15, pp. 439-458, in “Polymers in Nature” by E. A. MacGregor and C. T. Greenwood, published by John Wiley & Sons, Chapter 6, pp. 240-328, 1980, and in “Industrial Gums—Polysaccharides and their Derivatives”, edited by Roy L. Whistler, Second Edition, published by Academic Press Inc., the content of these three publications being included in their entirety in the present patent application by reference.

As examples of modified or unmodified, optionally hydrolysed saccharides, oligosaccharides and polysaccharides that may be used in the invention, mention may be made especially of glucans, modified or unmodified starches (such as those derived, for example, from cereals, for instance wheat, corn or rice, from vegetables, for instance haricot beans, from tubers, for instance potato or cassava), which are different from the starch betaine as described above, amylose, amylopectin, glycogen, dextrans, β-glucans, celluloses and derivatives thereof (methylcelluloses, hydroxyalkylcelluloses, ethylhydroxyethylcelluloses or carboxymethylcelluloses), fructosans, inulin, levan, mannans, xylans, lignins, arabans, galactans, galacturonans, chitin, glucoronoxylans, arabinoxylans, xyloglucans, galactomannans, glucomannans, pectic acids and pectins, alginic acid and alginates, arabino-galactans, carrageenans, agars, glycosaminoglucans, gum arabics, gum tragacanths, ghatti gums, karaya gums, carob gums, guar gums and xanthan gums.

Examples of amino acids that may be mentioned include cysteine, lysine, alanine, N-phenylalanine, arginine, glycine and leucine, and mixtures thereof. As modified or unmodified, optionally hydrolysed oligopeptides, peptides and proteins that may be used according to the invention, mention may be made especially of modified or unmodified wool or silk protein hydrolysates, and plant proteins such as wheat proteins.

Among the polyamino acids that may be used, mention may be made of polylysine.

Among the enzymes that may be used, mention may be made of laccases, peroxidases, lipases, proteases, glycosidases, dextranases, uricases and alkaline phosphatase.

Among the branched or unbranched fatty acids that are suitable in the present invention, mention may be made especially of C₈-C₃₀ carboxylic acids such as palmitic acid, oleic acid, linoleic acid, myristic acid, stearic acid and lauric acid, and mixtures thereof. The fatty alcohols that may be used in the present invention especially include C₈-C₃₀ alcohols such as, for example, palmityl alcohol, oleyl alcohol, linoleyl alcohol, myristyl alcohol, stearyl alcohol and lauryl alcohol.

For the purposes of the present invention, a wax is a lipophilic compound that is solid at room temperature (about 25° C), with a reversible solid/liquid change of state, having a melting point of greater than about 40° C. and which may be up to 200° C., and having an anisotropic crystal organization in the solid state. In general, the size of the wax crystals is such that crystals scatter and/or diffuse light, giving the composition comprising them a more or less opaque, cloudy appearance. By raising the wax to its melting point, it is possible to make it miscible with oils and to form a microscopically homogeneous mixture, but on returning the temperature of the mixture to room temperature, a microscopically and macroscopically detectable recrystallization of the wax in the oils of the mixture is obtained (opalescence).

As waxes that may be used in the present invention, mention may be made of waxes of animal origin such as beeswax, spermaceti, lanolin wax and lanolin derivatives; plant waxes such as carnauba wax, candelilla wax, ouricury wax, Japan wax, cocoa butter, cork fibre wax or sugar cane wax; mineral waxes, for example paraffin wax, petroleum jelly wax, lignite wax, microcrystalline waxes or ozokerites.

Among the ceramides that may be mentioned in particular are the ceramides of categories I, II, III and V according to the Dawning classification, and more particularly N-oleyldehydrosphingosine.

The hydroxylated organic acids are chosen from those that are well known and used in the art. Mention may be made especially of citric acid, lactic acid, tartaric acid and malic acid.

The UV-A-active and/or UV-B-active sunscreens that may be used according to the invention are those that are well known to those skilled in the art. Mention may be made especially of dibenzoylmethane derivatives such as 4-methyldibenzoylmethane, 4-isopropyldibenzoylmethane, 4-tert-butyldibenzoylmethane, 2,4-dimethyldibenzoylmethane, 4-tert-butyl-4′-diisopropyldibenzoylmethane, p-aminobenzoic acid and its esters such as 2-ethylhexyl p-dimethylaminobenzoate and N-propoxylated ethyl p-aminobenzoate, salicylates such as triethanolamine salicylate, cinnamic acid esters such as 2-ethylhexyl 4-methoxycinnamate, methyl diisopropylcinnamate, menthyl anthranilate, benzotriazole derivatives, triazine derivatives, β,β′-diphenylacrylate derivatives such as 2-ethylhexyl 2-cyano-3,3-diphenylacrylate and ethyl 2-cyano-3,3-diphenylacrylate, 2-phenylbenzimidazole-5-sulphonic acid and its salts, benzophenone derivatives, benzylidenecamphor derivatives, silicone screening agents, etc.

As examples of antioxidants and free-radical scavengers that may be used in the present invention, mention may be made of ascorbic acid, ascorbyl compounds such as ascorbyl dipalmitate, t-butylhydroquinone, polyphenols such as phloroglucinol, sodium sulphite, erythorbic acid and flavonoids.

The chelating agents may be chosen especially from EDTA (ethylenediaminetetraacetic acid) and its salts such as disodium EDTA and dipotassium EDTA, phosphate compounds such as sodium metaphosphate, sodium hexametaphosphate and tetrapotassium pyrophosphate, and phosphonic acids and salts thereof, such as ethylenediaminetetramethylenephosphonic acid salts.

The antidandruff agents are chosen, for example, from:
benzethonium chloride, benzalkonium chloride, chlorhexidine, chloramine T, chloramine B, 1,3-dibromo-5,5-dimethylhydantoin, 1,3-dichloro-5,5-dimethylhydantoin, 3-bromo-1-chloro-5,5-dimethyl-hydantoin and N-chlorosuccinimide;
1-hydroxy-2-pyridone derivatives such as, for example, 1-hydroxy-4-methyl-2-pyridone, 1-hydroxy-6-methyl-2-pyridone and 1-hydroxy-4,6-dimethyl-2-pyridone;
trihalocarbamides;
triclosan;
azole compounds such as climbazole, ketoconazole, clotrinazole, econazole, isoconazole and miconazole b;
antifungal polymers such as amphotericin B or nystatin;
selenium sulphides;
sulphur in its various forms, cadmium sulphide, allantoin, coal tar or wood tar and in particular derivatives thereof, cade oil, undecylenic acid, fumaric acid and allylamines such as terbinafine.

The said agents may also be used in the form of the addition salts thereof with physiologically acceptable acids, especially in the form of the sulphuric acid, nitric acid, thiocyanic acid, hydrochloric acid, hydrobromic acid, hydriodic acid, phosphoric acid, acetic acid, benzoic acid, glycolic acid, aceturic acid, succinic acid, nicotinic acid, tartaric acid, maleic acid, palmitic acid, methanesulphonic acid, propanoic acid, 2-oxopropanoic acid, propanedioic acid, 2-hydroxy-1,4-butanedioic acid, 3-phenyl-2-propenoic acid, α-hydroxybenzeneacetic acid, ethanesulphonic acid, 2-hydroxyethanesulphonic acid, 4-methylbenzenesulphonic acid, 4-amino-2-hydroxybenzoic acid, 2-phenoxybenzoic acid, 2-acetyloxybenzoic acid, picric acid, lactic acid, citric acid, malic acid, oxalic acid and amino acid salts.

The antidandruff agents mentioned above may also, where appropriate, be used in the form of the addition salts thereof with physiologically acceptable organic or mineral bases. Examples of organic bases are especially low molecular weight alkanolamines such as ethanolamine, diethanolamine, N-ethylethanolamine, triethanolamine, diethylaminoethanol and 2-amino-2-methylpropanedione; non-volatile bases such as ethylenediamine, hexamethylenediamine, cyclohexylamine, benzylamine and N-methylpiperazine; quaternary ammonium hydroxides, for example trimethylbenzyl hydroxide; guanidine and its derivatives, and particularly its alkyl derivatives. Examples of mineral bases are especially the alkali metal salts, for instance the sodium or potassium salts; the ammonium salts, the alkaline-earth metal salts, for instance the magnesium or calcium salts; the salts of cationic di-, tri- or tetravalent metals, for instance the zinc, aluminium and zirconium salts. Alkanolamines, ethylenediamine and mineral bases such as the alkali metal salts are preferred.

The seborrhoea regulators are, for example, succinylchitosan and poly-β-alanine.

The calmants are, for example, azulene and glycyrrhetinic acid.

The cationic surfactants are those that are well known per se, such as optionally polyoxyalkylenated primary, secondary or tertiary fatty amine salts; quaternary ammonium salts such as tetraalkylammonium, alkylamidoalkyltrialkylammonium, trialkylbenzylammonium, trialkylhydroxyalkylammonium or alkylpyridinium chlorides or bromides; imidazoline derivatives.

The term “cationic polymer” means any polymer containing cationic groups and/or groups that may be ionized into cationic groups.

The cationic polymers that may be used in accordance with the present invention may be chosen from any of those already known per se as improving the cosmetic properties of hair treated with detergent compositions, i.e. especially those described in patent application EP-A-0 337 354 and in French patent applications FR-A-2 270 846, 2 383 660, 2 598 611, 2 470 596 and 2 519 863.

The preferred cationic polymers are chosen from those containing units comprising primary, secondary, tertiary and/or quaternary amine groups that may either form part of the main polymer chain or may be borne by a side substituent that is directly attached thereto.

The cationic polymers used generally have a number-average molecular mass of between 500 and 5×10⁶ approximately and preferably between 10³ and 3×10⁶ approximately.

Among the cationic polymers that may be mentioned more particularly are polymers of the polyamine, polyamino amide and polyquaternary ammonium type. These are known products.

The polymers of the polyamine, polyamino amide and polyquaternary ammonium type that may be used in the composition of the present invention are those described in French patents 2 505 348 and 2 542 997. Among these polymers, mention may be made of:

(1) homopolymers or copolymers derived from acrylic acid or methacrylic acid esters or amides;

(2) the cellulose ether derivatives containing quaternary ammonium groups, described in French patent 1 492 597;

(3) cationic cellulose derivatives such as cellulose copolymers or cellulose derivatives grafted with a water-soluble quaternary ammonium monomer, and described in particular in U.S. Pat. No. 4,131,576, such as hydroxyalkylcelluloses, for instance hydroxymethyl-, hydroxyethyl- or hydroxypropylcelluloses grafted, in particular, with a methacryloylethyltrimethylammonium, methacrylamidopropyltrimethylammonium or dimethyl-diallylammonium salt;

(4) the cationic polysaccharides described more particularly in U.S. Pat. Nos. 3,589,578 and 4,031,307, such as guar gums containing cationic trialkylammonium groups;

(5) polymers consisting of piperazinyl units and of divalent alkylene or hydroxyalkylene groups containing straight or branched chains, optionally interrupted by oxygen, sulphur or nitrogen atoms or by aromatic or heterocyclic rings, as well as the oxidation and/or quaternization products of these polymers. Such polymers are described, in particular, in French patents 2 162 025 and 2 280 361;

(6) water-soluble polyamino amides such as those described, in particular, in French patents 2 252 840 and 2 368 508;

(7) polyamino amide derivatives, for example, adipic acid/dialkylaminohydroxyalkyldialkylenetriamine polymers in which the alkyl group contains from 1 to 4 carbon atoms and preferably denotes a methyl, ethyl or propyl group, and the alkylene group contains from 1 to 4 carbon atoms and preferably denotes an ethylene group. Such polymers are described in particular in French patent 1 583 363.

(8) polymers obtained by reaction of a polyalkylene polyamine containing two primary amine groups and at least one secondary amine group with a dicarboxylic acid chosen from diglycolic acid and saturated aliphatic dicarboxylic acids having from 3 to 8 carbon atoms. The molar ratio between the polyalkylene polyamine and the dicarboxylic acid is between 0.8:1 and 1.4:1; the polyamino amide resulting therefrom is reacted with epichlorohydrin in a molar ratio of epichlorohydrin relative to the secondary amine group of the polyamino amide of between 0.5:1 and 1.8:1. Such polymers are described in particular in U.S. Pat. Nos. 3,227,615 and 2,961,347;

(9) cyclopolymers of alkyldiallylamine or of dialkyldiallylammonium such as the dimethyldiallyl-ammonium chloride homopolymer and copolymers of diallyldimethylammonium chloride and of acrylamide;

(10) quaternary diammonium polymers with a number-average molecular mass generally of between 1000 and 100 000 such as those described, for example, in French patents 2 320 330, 2 270 846, 2 316 271, 2 336 434 and 2 413 907 and U.S. Pat. Nos. 2,273,780, 2,375,853, 2,388,614, 2,454,547, 3,206,462, 2,261,002, 2,271,378, 3,874,870, 4,001,432, 3,929,990, 3,966,904, 4,005,193, 4,025,617, 4,025,627, 4,025,653, 4,026,945 and 4,027,020;

(11) polyquaternary ammonium polymers such as those especially described in patent application EP-A-122 324;

(12) quaternary polymers of vinylpyrrolidone and of vinylimidazole, such as, for example, the products sold under the names Luviquat® FC 905, FC 550 and FC 370 by the company BASF;

(13) polyamines such as Polyquart® H sold by Henkel under the reference name “Polyethylene glycol (15) Tallow polyamine” in the CTFA dictionary;

(14) crosslinked (meth)acryloyloxy(C₁-C₄)alkyltri(C₁-C₄)alkylammonium salt polymers such as those sold under the name Salcaree SC 92, Salcareo SC 95 and Salcare® SC 96 by the company Allied Colloids; and

mixtures thereof.

Other cationic polymers that may be used in the context of the invention are cationic proteins or cationic protein hydrolysates, polyalkyleneimines, in particular polyethyleneimines, polymers containing vinylpyridine or vinylpyridinium units, condensates of polyamines and of epichlorohydrin, quaternary polyureylenes and chitin derivatives.

The amphoteric polymers that may be used in accordance with the invention may be chosen from polymers comprising units B and C randomly distributed in the polymer chain, in which B denotes a unit derived from a monomer comprising at least one basic nitrogen atom and C denotes a unit derived from an acidic monomer comprising one or more carboxylic or sulphonic groups, or alternatively B and C may denote groups derived from zwitterionic carboxybetaine or sulphobetaine monomers; B and C may also denote a cationic polymer chain comprising primary, secondary, tertiary or quaternary amine groups, in which at least one of the amine groups bears a carboxylic or sulphonic group linked via a hydrocarbon-based radical, or alternatively B and C form part of a chain of a polymer containing an ethylene-dicarboxylic unit in which one of the carboxylic groups has been made to react with a polyamine comprising one or more primary or secondary amine groups.

The amphoteric polymers corresponding to the above definition that are more particularly preferred are chosen from the following polymers:

(1) polymers resulting from the copolymerization of a monomer derived from a vinyl compound bearing a carboxylic group such as, more particularly, acrylic acid, methacrylic acid, maleic acid, α-chloroacrylic acid, and a basic monomer derived from a substituted vinyl compound containing at least one basic atom, such as, more particularly, dialkylaminoalkyl methacrylate and acrylate, dialkylaminoalkylmethacrylamide and -acrylamide. Such compounds are described in U.S. Pat. No. 3,836,537. Mention may also be made of the sodium acrylate/acrylamidopropyltrimethylammonium chloride copolymer sold under the name Polyquart® KE 3033 by the company Henkel.

The vinyl compound may also be a dialkyl-diallylammonium salt such as diethyldiallylammonium chloride.

The copolymers of acrylic acid and of the latter monomer are sold under the names Merquat® 280, Merquat® 295 and Merquat® Plus 3330 by the company Calgon.

(2) Polymers containing units derived from:
a) at least one monomer chosen from acrylamides and methacrylamides substituted on the nitrogen with an alkyl radical,
b) at least one acidic comonomer containing one or more reactive carboxylic groups, and
c) at least one basic comonomer such as esters containing primary, secondary, tertiary and quaternary amine substituents of acrylic and methacrylic acids and the product of quaternization of dimethylaminoethyl methacrylate with dimethyl or diethyl sulphate.

The N-substituted acrylamides or methacrylamides which are more particularly preferred according to the invention are groups in which the alkyl radicals contain from 2 to 12 carbon atoms and more particularly N-ethylacrylamide, N-tert-butylacrylamide, N-tert-octylacrylamide, N-octylacrylamide, N-decylacrylamide, N-dodecylacrylamide and the corresponding methacrylamides.

The acidic comonomers are chosen more particularly from acrylic acid, methacrylic acid, crotonic acid, itaconic acid, maleic acid and fumaric acid and alkyl monoesters, having 1 to 4 carbon atoms, of maleic or fumaric acids or anhydrides. The preferred basic comonomers are aminoethyl, butylaminoethyl, N,N′-dimethylaminoethyl and N-tert-butylaminoethyl methacrylates. The copolymers whose CTFA (4th edition, 1991) name is octylacrylamide/acrylates/butylaminoethyl methacrylate copolymer such as the products sold under the name Amphomer® or Lovocryl® 47 by the company National Starch are particularly used.

(3) Crosslinked and alkylated polyamino amides partially or totally derived from polyamino amides of general formula:
—[—CO—R₁₀—C—O-Z-]- (II)
in which R₁₀ represents a divalent radical derived from a saturated dicarboxylic acid, a mono- or dicarboxylic aliphatic acid containing an ethylenic double bond, an ester of a lower alkanol, having 1 to 6 carbon atoms, of these acids or a radical derived from the addition of any one of the said acids to a bis(primary) or bis(secondary) amine, and Z denotes a bis(primary), mono- or bis(secondary) polyalkylene-polyamine radical and preferably represents:

a) in proportions of from 60 to 100 mol %, the radical
—NH—[—(CH₂)ₓ—NH—]ₚ— (III)
where x=2 and p=2 or 3, or alternatively x=3 and p=2 this radical being derived from diethylenetriamine, from triethylenetetraamine or from dipropylenetriamine;

b) in proportions of from 0 to 40 mol %, the radical (III) above in which x=2 and p=1 and which is derived from ethylenediamine, or the radical derived from piperazine

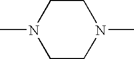

c) in proportions of from 0 to 20 mol %, the —NH—(CH₂)₆—NH— radical derived from hexamethylene-diamine, these polyamino amines being crosslinked by addition of a difunctional crosslinking agent chosen from epihalohydrins, diepoxides, dianhydrides and bis-unsaturated derivatives, using from 0.025 to 0.35 mol of crosslinking agent per amine group of the polyamino amide and alkylated by the action of acrylic acid, chloroacetic acid or an alkane sultone, or salts thereof.

The saturated carboxylic acids are preferably chosen from acids having 6 to 10 carbon atoms, such as adipic acid, 2,2,4-trimethyladipic acid and 2,4,4-trimethyladipic acid, terephthalic acid and acids containing an ethylenic double bond such as, for example, acrylic acid, methacrylic acid and itaconic acid. The alkane sultones used in the alkylation are preferably propane sultone or butane sultone, and the salts of the alkylating agents are preferably the sodium or potassium salts.

(4) Polymers containing zwitterionic units of formula: 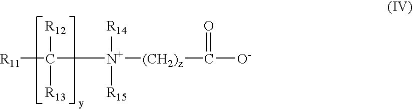
in which R₁₁ denotes a polymerizable unsaturated group such as an acrylate, methacrylate, acrylamide or methacrylamide group, y and z each represent an integer from 1 to 3, R₁₂ and R₁₃ represent a hydrogen atom, a methyl, ethyl or propyl group, R₁₄ and R₁₅ represent a hydrogen atom or an alkyl radical such that the sum of the carbon atoms in R₁₄ and R₁₅ does not exceed 10.

The polymers comprising such units can also contain units derived from non-zwitterionic monomers such as dimethyl or diethylaminoethyl acrylate or methacrylate or alkyl acrylates or methacrylates, acrylamides or methacrylamides or vinyl acetate.

By way of example, mention may be made of the copolymer of methyl methacrylate/dimethyl carboxymethylammonio ethyl methacrylate such as the product sold under the name Diaformer® Z301 by the company Sandoz.

(5) Polymers derived from chitosan containing monomer units corresponding to the following formulae: 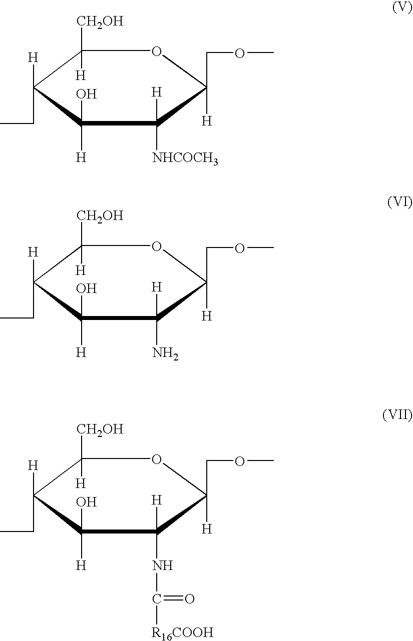
the unit (V) being present in proportions of between 0 and 30%, the unit (VI) in proportions of between 5 and 50% and the unit (VII) in proportions of between 30 and 90%, it being understood that, in this unit F, R₁₆ represents a radical of formula: 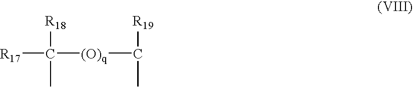
in which if q=0, R₁₇, R₁₈ and R₁₉, which may be identical or different, each represent a hydrogen atom, a methyl, hydroxyl, acetoxy or amino residue, a monoalkylamine residue or a dialkylamine residue which are optionally interrupted by one or more nitrogen atoms and/or optionally substituted with one or more amine, hydroxyl, carboxyl, alkylthio or sulphonic radicals, an alkylthio residue in which the alkyl radical bears an amino residue, at least one of the radicals R₁₇, R₁₈ and R₁₉ being, in this case, a hydrogen atom; or, if q=1, R₁₇, R₁₈ and R₁₉ each represent a hydrogen atom, as well as the salts formed by these compounds with bases or acids.

(6) polymers derived from the N-carboxyalkylation of chitosan, such as N-carboxymethylchitosan or N-carboxybutylchitosan sold under the name “Evalsan®” by the company Jan Dekker.

(7) Polymers corresponding to the general formula (IX) as described, for example, in French patent 1 400 366: 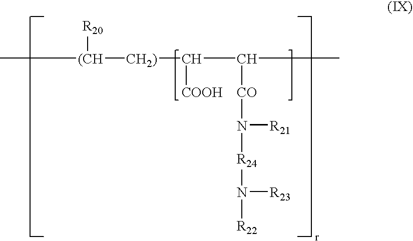
in which R₂₀ represents a hydrogen atom, a CH₃O, CH₃CH₂O or phenyl radical, R₂₁ denotes hydrogen or a lower alkyl radical such as methyl or ethyl, R₂₂ denotes hydrogen or a lower alkyl radical such as methyl or ethyl, R₂₃ denotes a lower alkyl radical such as methyl or ethyl or a radical corresponding to the formula: —R₂₄—N(R₂₂)₂, R₂₄ representing a —CH₂—CH₂—, —CH₂—CH₂—CH₂— or —CH₂—CH(CH₃)— group, R₂₂ having the meanings mentioned above, as well as the higher homologues of these radicals and containing up to 6 carbon atoms. (8) Amphoteric polymers of the type -D-X-D-X— chosen from:

a) polymers obtained by the action of chloroacetic acid or sodium chloroacetate on compounds containing at least one unit of formula:
-D-X-D-X-D- (X)
where D denotes a radical 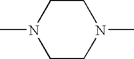
and X denotes the symbol E or E′, E or E′, which may be identical or different, denotes a divalent radical which is an alkylene radical with a straight or branched chain containing up to 7 carbon atoms in the main chain, which is unsubstituted or substituted with hydroxyl groups and which can contain, in addition to the oxygen, nitrogen and sulphur atoms, 1 to 3 aromatic and/or heterocyclic rings; the oxygen, nitrogen and sulphur atoms being present in the form of ether, thioether, sulphoxide, sulphone, sulphonium, alkylamine or alkenylamine groups, hydroxyl, benzylamine, amine oxide, quaternary ammonium, amide, imide, alcohol, ester and/or urethane groups;

b) polymers of formula:
-D-X-D-X- (XI)
where D denotes a radical 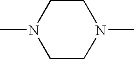
and X denotes the symbol E or E′ and at least once E′; E having the meaning given above and E′ being a divalent radical which is an alkylene radical with a straight or branched chain having up to 7 carbon atoms in the main chain, which is unsubstituted or substituted with one or more hydroxyl radicals and containing one or more nitrogen atoms, the nitrogen atom being substituted with an alkyl chain which is optionally interrupted by an oxygen atom and necessarily containing one or more carboxyl functions or one or more hydroxyl functions and betainized by reaction with chloroacetic acid or sodium chloroacetate.

(9) (C₁-C₅)alkyl vinyl ether/maleic anhydride copolymers partially modified by semiamidation with an N,N-dialkylaminoalkylamine such as N,N-dimethylamino-propylamine or by semiesterification with an N,N-dialkanolamine. These copolymers can also contain other vinyl comonomers such as vinylcaprolactam.

The silicones that may be used in accordance with the invention may be soluble or insoluble in water, and they may be in particular polyorganosiloxanes that are insoluble in water; they may be in the form of oils, waxes, resins or gums.

The organopolysiloxanes are defined in greater detail in Walter Noll's “Chemistry and Technology of Silicones” (1968) Academic Press. They can be volatile or non-volatile.

When they are volatile, the silicones are more particularly chosen from those having a boiling point of between 60° C. and 260° C., and even more particularly from:

(i) cyclic silicones containing from 3 to 7 and preferably from 4 to 5 silicon atoms. These are, for example, octamethylcyclotetrasiloxane sold in particular under the name “Volatile Silicone 7207” by Union Carbide or “Silbione 70045 V 2” by Rhodia, decamethylcyclopentasiloxane sold under the name “Volatile Silicone 7158” by Union Carbide or “Silbione 70045 V 5” by Rhodia.

Mention may also be made of cyclocopolymers of the dimethylsiloxane/methylalkylsiloxane type, such as “Volatile Silicone FZ 3109” sold by the company Union Carbide, having the chemical structure:

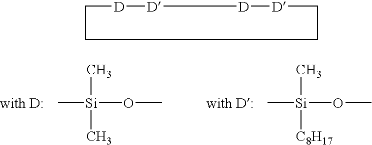

(ii) linear volatile silicones containing 2 to 9 silicon atoms and having a viscosity of less than or equal to 5×10⁻⁶ m²/s at 25° C. An example is decamethyltetrasiloxane sold in particular under the name “SH 200” by the company Toray Silicone. Silicones belonging to this category are also described in the article published in Cosmetics and Toiletries, Vol. 91, January 76, pp. 27-32, Todd & Byers “Volatile Silicone Fluids for Cosmetics”.

Among the non-volatile silicones that may be mentioned especially are polyalkylsiloxanes, polyarylsiloxanes, polyalkylarylsiloxanes, silicone gums and resins, and polyorganosiloxanes modified with organofunctional groups.

The organomodified silicones that may be used in accordance with the invention are silicones as defined above and containing in their structure one or more organofunctional groups attached via a hydrocarbon-based group.

Among the organomodified silicones that may be mentioned are polyorganosiloxanes containing:

polyethyleneoxy and/or polypropyleneoxy groups optionally containing C₆-C₂₄ alkyl groups, such as the products known as dimethicone copolyol sold by the company Dow Corning under the name DC 1248 or the oils Silwet® L 722, L 7500, L 77 and L 711 from the company Union Carbide and the (C₁₂)alkylmethicone copolyol sold by the company Dow Corning under the name Q2 5200;

substituted or unsubstituted amine groups, such as the products sold under the name GP 4 Silicone Fluid and GP 7100 by the company Genesee, or the products sold under the names Q2 8220 and Dow Corning 929 or 939 by the company Dow Corning. The substituted amine groups are, in particular, C₁-C₄ aminoalkyl groups;

thiol groups such as the products sold under the names “GP 72 A” and “GP 71” from Genesee;

alkoxylated groups such as the product sold under the name “Silicone Copolymer F-755” by SWS Silicones and Abil Wax® 2428, 2434 and 2440 by the company Goldschmidt;

hydroxylated groups such as the polyorganosiloxanes containing a hydroxyalkyl function, described in French patent application FR-A-85/16334;

acyloxyalkyl groups such as, for example, the polyorganosiloxanes described in patent U.S. Pat. No. 4,957,732;

anionic groups of the carboxylic acid type, such as, for example, in the products described in patent EP 186 507 from the company Chisso Corporation, or of alkylcarboxylic type, such as those present in the product X-22-3701E from the company Shin-Etsu; 2-hydroxyalkyl sulphonate; 2-hydroxyalkyl thiosulphate such as the products sold by the company Goldschmidt under the names “Abil® S201” and “Abil® S255”.

hydroxyacylamino groups, such as the polyorganosiloxanes described in patent application EP 342 834. Mention may be made, for example, of the product Q2-8413 from the company Dow Corning.

Mention may be made especially, as oils of plant origin, of sweet almond oil, avocado oil, castor oil, olive oil, jojoba oil, sunflower oil, wheatgerm oil, sesame seed oil, groundnut oil, grapeseed oil, soybean oil, rapeseed oil, safflower oil, coconut oil, corn oil, hazelnut oil, karite butter, palm oil, apricot kernel oil or beauty-leaf oil; as an oil of animal origin, of perhydrosqualene; as oils of mineral origin, of liquid paraffin and liquid petroleum jelly.

The polyisobutenes and poly(α-olefins) are chosen from those that are well known in the art.

Examples of esters that may especially be mentioned are fatty acid esters, for instance isopropyl myristate, isopropyl palmitate, 2-ethylhexyl palmitate, purcellin oil (stearyl octanoate), isononyl isononanoate, isostearyl isononanoate and isopropyl lanolate, and mixtures thereof.

The anionic polymers generally used in the present invention are polymers comprising groups derived from carboxylic acids, sulphonic acids or phosphoric acids, and having a weight-average molecular mass of between 500 and 5 000 000.

The carboxylic groups are provided by unsaturated monocarboxylic or dicarboxylic acid monomers such as those corresponding to the formula: 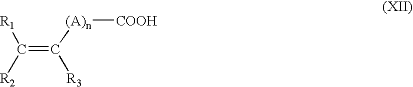
in which n is an integer from 0 to 10, A denotes a methylene group, optionally connected to the carbon atom of the unsaturated group or to the neighbouring methylene group when n is greater than 1 via a hetero atom such as oxygen or sulphur, R₁ denotes a hydrogen atom or a phenyl or benzyl group, R₂ denotes a hydrogen atom or a lower alkyl or carboxyl group, and R₃ denotes a hydrogen atom, a lower alkyl group or a —CH₂—COOH, phenyl or benzyl group.

In formula (XII) above, a lower alkyl group preferably contains from 1 to 4 carbon atoms and in particular denotes methyl and ethyl groups.

The anionic polymers containing carboxylic groups that are preferred according to the invention are:

A) homo- or copolymers of acrylic or methacrylic acid or salts thereof and in particular the products sold under the names Versicol® E or K by the company Allied Colloid, Ultrahold® by the company BASF, the copolymers of acrylic acid and acrylamide sold in the form of their sodium salt under the names Reten® 421, 423 or 425 by the company Hercules, and the sodium salts of polyhydroxycarboxylic acids;

B) copolymers of acrylic or methacrylic acids with a monoethylenic monomer such as ethylene, styrene, vinyl esters and acrylic or methacrylic acid esters, optionally grafted onto a polyalkylene glycol such as polyethylene glycol and optionally crosslinked. Such polymers are described in particular in French patent 1 222 944 and German patent application No. 2 330 956. Mention may be made in particular of copolymers whose chain comprises an optionally N-alkylated and/or hydroxyalkylated acrylamide unit, such as those described in particular in the Luxembourg patent applications 75370 and 75371 or sold under the name Quadramer® by the company American Cyanamid. Mention may also be made of copolymers of acrylic acid and of C₁-C₄ alkyl methacrylate and the copolymer of methacrylic acid and of ethyl acrylate sold under the name Luvimer® MAEX by the company BASF;

C) copolymers derived from crotonic acid, such as those whose chain comprises vinyl acetate or propionate units and optionally other monomers such as allylic or methallylic esters, vinyl ether or vinyl ester of a saturated, linear or branched carboxylic acid containing a long hydrocarbon-based chain such as those comprising at least 5 carbon atoms, it being possible for these polymers to be grafted and crosslinked, or alternatively a vinyl, allylic or methallylic ester of an α- or β-cyclic carboxylic acid. Such polymers are described, inter alia, in French patents 1 222 944, 1 580 545, 2 265 782, 2 265 781, 1 564 110 and 2 439 798. Commercial products falling within this category are the resins 28-29-30, 26-13-14 and 28-13-10 sold by the company National Starch;

D) polymers derived from maleic, fumaric or itaconic acids or anhydrides with vinyl esters, vinyl ethers, vinyl halides, phenylvinyl derivatives or acrylic acid and its esters; these polymers may be esterified. Such polymers are described in particular in U.S. Pat. Nos. 2,047,398, 2,723,248 and 2,102,113 and GB patent 839 805, and especially those sold under the names Gantrez® AN or ES by the company ISP.

Polymers also falling within this category are the copolymers of maleic, citraconic or itaconic anhydrides and of an allylic or methallylic ester optionally comprising an acrylamide or methacrylamide group, an α-olefin, acrylic or methacrylic esters, acrylic or methacrylic acid or vinylpyrrolidone in their chain, the anhydride functions being monoesterified or monoamidated. These polymers are described, for example, in French patents 2 350 384 and 2 357 241 by the Applicant;

E) polyacrylamides comprising carboxylate groups.

The polymers comprising sulphonic groups are polymers comprising vinylsulphonic, styrenesulphonic, naphthalenesulphonic or acrylamidoalkylsulphonic units.

These polymers can be chosen in particular from:

polyvinylsulphonic acid salts with a molecular weight of between about 1000 and 100 000, as well as copolymers with an unsaturated comonomer such as acrylic or methacrylic acids and esters thereof, as well as acrylamide or derivatives thereof, vinyl ethers and vinylpyrrolidone;

polystyrenesulphonic acid salts, the sodium salts having a molecular weight of about 500 000 and of about 100 000, sold, respectively, under the names Flexan® 500 and Flexan® 130 by National Starch. These compounds are described in patent FR 2 198 719;

polyacrylamidesulphonic acid salts such as those mentioned in U.S. Pat. No. 4,128,631 and more particularly polyacrylamidoethylpropanesulphonic acid sold under the name Cosmedia Polymer® HSP 1180 by Henkel.

According to the invention, the anionic polymers are preferably chosen from acrylic acid copolymers such as the acrylic acid/ethyl acrylate/N-tert-butylacrylamide terpolymer sold under the name Ultrahold Strong® by the company BASF, copolymers derived from crotonic acid, such as the vinyl acetate/vinyl tert-butylbenzoate/crotonic acid terpolymers and the crotonic acid/vinyl acetate/vinyl neododecanoate terpolymers sold under the name Resin 28-29-30 by the company National Starch, polymers derived from maleic, fumaric or itaconic acids with vinyl esters, vinyl ethers, vinyl halides, phenylvinyl derivatives or acrylic acid and esters thereof, such as the methyl vinyl ether/monoesterified maleic anhydride copolymer sold under the name Gantrez® ES 425 by the company ISP, the copolymers of methacrylic acid and of methyl methacrylate sold under the name Eudragit® L by the company Rohm Pharma, the copolymer of methacrylic acid and of ethyl acrylate sold under the name Luvimere MAEX by the company BASF, the vinyl acetate/crotonic acid copolymer sold under the name Luviset® CA 66 by the company BASF and the vinyl acetate/crotonic acid/polyethylene glycol terpolymer sold under the name Aristoflex® A by the company BASF.

According to the invention, the anionic polymers may also be used in the form of a latex or pseudolatex, i.e. in the form of an aqueous dispersion of insoluble polymer particles.

As nonionic polymers that may be used according to the present invention, mention may be made especially of:

vinylpyrrolidone homopolymers;

copolymers of vinylpyrrolidone and vinyl acetate;

polyalkyloxazolines such as the polyethyloxazolines sold by the company Dow Chemical under the names Peox® 50 000, Peox® 200 000 and Peox® 500 000;

vinyl acetate homopolymers, such as the product sold under the name Appretan® EM by the company Hoechst, or the product sold under the name Rhodopas® A 012 by the company Rhône-Poulenc;

copolymers of vinyl acetate and acrylic ester, such as the product sold under the name Rhodopas® AD 310 by Rhône-Poulenc;

copolymers of vinyl acetate and ethylene, such as the product sold under the name Appretan® TV by the company Hoechst;

copolymers of vinyl acetate and maleic ester, for example of dibutyl maleate, such as the product sold under the name Appretan® MB Extra by the company Hoechst;

copolymers of polyethylene and maleic anhydride;

alkyl acrylate homopolymers and alkyl methacrylate homopolymers, such as the product sold under the name Micropearl® RQ 750 by the company Matsumoto or the product sold under the name Luhydrane A 848 S by the company BASF;

acrylic ester copolymers such as, for example, copolymers of alkyl acrylates and alkyl methacrylates, such as the products sold by the company Rohm & Haas under the names Primal® AC-261 K and Eudragit® NE 30 D, by the company BASF under the names Acronal® 601, Luhydran® LR 8833 or 8845, and by the company Hoechst under the names Appretan® N 9213 or N 921 2;

copolymers of acrylonitrile and a nonionic monomer chosen, for example, from butadiene and alkyl (meth)acrylates; mention may be made of the products sold under the names Nipol® LX 531 8 by the company Nippon Zeon or those sold under the name CJ 0601 8 by the company Rohm & Haas;

polyurethanes, such as the products sold under the names Acrysol® RM 1020 or Acrysol® RM 2020 by the company Rohm & Haas, and the products Uraflex® XP 401 UZ and Uraflex® XP 402 UZ by the company DSM Resins;

copolymers of alkyl acetate and urethane, such as the product 8538-33 by the company National Starch;

polyamides, such as the product Estapor® LO 11 sold by the company Rhône-Poulenc;

unmodified or chemically modified nonionic guar gums.

The unmodified nonionic guar gums are, for example, the products sold under the name Vidogum® GH 175 by the company Unipectine and under the name Jaguar® C by the company Meyhall.

The modified nonionic guar gums that can be used according to the invention are preferably modified with C₁-C₆ hydroxyalkyl groups. Mention may be made, for example, of hydroxymethyl, hydroxyethyl, hydroxypropyl and hydroxybutyl groups.

These guar gums are well known in the state of the art and can be prepared, for example, by reacting corresponding alkene oxides, such as, for example, propylene oxides, with guar gum so as to obtain a guar gum modified with hydroxypropyl groups.

Such nonionic guar gums optionally modified with hydroxyalkyl groups are sold, for example, under the trade names Jaguar® HP8, Jaguar® HP60 and Jaguar® HP120, Jaguar® DC 293 and Jaguar® HP 105 by the company Meyhall or under the name Galactasol® 4H4FD2 by the company Aqualon.

The alkyl groups of the nonionic polymers preferably contain from 1 to 6 carbon atoms.

The reducing agents may be chosen from thio acids and salts thereof (thioglycolic acid or thiolactic acid, cysteine or cysteamine), alkali metal sulphites or alkaline-earth metal sulphites, reducing sugars such as glucose, vitamin C and its derivatives, sulphinic acid derivatives and phosphines.

The amounts of these various cosmetic active agents are those that are conventionally used in the fields under consideration, for example from 0.001% to 30% by weight relative to the total weight of the composition.

The composition according to the invention may also comprise a cosmetically acceptable solvent or mixture of solvents.

For the purposes of the invention, the expression “cosmetically acceptable” means solvents that are physiologically acceptable and non-toxic, and which may be applied to human skin or hair.

As solvents that may be used according to the invention, mention may be made especially of water, alcohols, preferably C₁-C₆ lower alkanols and especially ethanol, propanol and isopropanol, polyols such as propylene glycol, hexylene glycol, glycerol and pentanediol, and polyol ethers, and mixtures thereof.

The solvent(s) generally represent(s) 0.1% to 99% of the total weight of the composition.

The pH of the compositions according to the invention is preferably between 2 and 12 and even more preferably between 3 and 11.

The composition of the invention may also contain conventional adjuvants such as fragrances, fillers, pH regulators (acidic or basic) and preserving agents.

The amounts of these various adjuvants in the composition according to the invention are those that are conventionally used in cosmetics.

Needless to say, a person skilled in the art will take care to select the optional additional adjuvants and/or the amount thereof such that the advantageous properties of the composition according to the invention are not, or are not substantially, adversely affected by the envisaged addition.

A subject of the present invention is also a process for treating keratin materials, comprising the application to the said keratin materials of a composition as defined above, and the irradiation of this composition to obtain a crosslinked deposit.

In particular, the present invention relates to a process for treating keratin materials, comprising the following steps:

a) applying to the keratin materials a cosmetic composition according to the invention as defined above, and

b) irradiating the said composition on the keratin materials to crosslink the composition.

The composition may be applied to wet or dry, clean or dirty keratin materials. The keratin materials are preferably dried after applying the composition and before irradiating.

An acting time at room temperature or with heat, or under a red lamp, may also be included in the process, before or after step b).

Finally, the process may include, before the irradiation step b), a step of partial masking of the keratin materials, and, after the irradiation step b), a rinsing step, or, when the keratin materials are hair, a shampooing or styling step.

Patterns may also be made on the keratin materials. In the case of making such patterns, the process preferably includes a drying step before the step of partial masking of the said keratin materials and after the step of applying the composition. Thus, in practice, drying is performed after applying the composition, and a portion of the substrate is then masked, for example with aluminium foil, and the whole area is irradiated. Rinsing is then performed. In the case of the hair, this rinsing may be followed by shampooing.

This masking technique makes it possible in particular to obtain localized cosmetic effects by means of the subsequent action of cosmetic active agents (for example colorants) that have selective affinity for the irradiated areas.

The irradiation is preferably irradiation with radiation in the UVA range or in the visible range, and even more preferably UVA radiation.

For the purposes of the present invention, the expression “irradiation in the visible range” means either an irradiation performed with a suitable device (laser or lamps), or the simple action of sunlight.

When the keratin materials are hair, the process according to the invention may include, before or after the irradiation step b), a step of shaping the hair, for example using clips, rollers, etc.

When the compound(s) comprising at least one photodimerizable group according to the invention is (are) also heat-dimerizable, the various processes described above may be performed by replacing the irradiation step with a step of heating between 40 and 200° C.

The keratin materials are advantageously hair or nails.

The example that follows illustrates the invention without, however, limiting its scope. Unless otherwise specified, the amounts are given as percentages by mass.

### EXAMPLE

An aqueous dispersion D of a polymer of partially saponified polyvinyl acetate type bearing stilbazolium groups, adsorbed on to polyvinyl acetate particles, is used.

This dispersion has a solids content of 40%.

This aqueous dispersion D is used to make the following composition:

| | | |
|---|---|---|
| Dispersion D | 11.25 | g |
| Dimethicone copolyol | 0.5 | g |
| (sold by Zshimmer Schwarz under | | |
| the name Skinotan S10) | | |
| Water | qs 100 | g |

0.3 g of this composition is applied per g of hair.

The lock is massaged, combed and dried with a hairdryer.

The lock is irradiated at 360 nm for ten seconds for a total energy of 50 mJ/cm², or placed in daylight for 15 minutes.

A deposit on the lock, which is resistant to shampooing at least five times successively, is noted.

1-34. (canceled)

35. A cosmetic composition for treating keratin materials, comprising:
at least one cosmetic active agent and at least one compound having at least one photodimerizable group,
wherein the compound having the photodimerizable group has a molecular mass of greater than 500 g/mol.

36. The cosmetic composition according to claim 35, wherein the photodimerizable group is a monovalent radical selected from the group consisting of formulae: 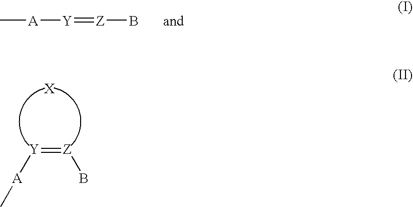
in which:
Y and Z denote, independently of each other, a carbon or nitrogen atom;
A is a divalent bonding group selected from the group consisting of an alkylene radical, a cycloaromatic radical, a heterocyclic radical, a carbonyl radical, an alkenylene radical, and combinations thereof;
B is a monovalent group selected from the group consisting of an alkyl radical, a cycloaromatic radical, a heterocyclic radical, a carbonyl radical, an alkenyl radical, and combinations thereof;
X is a divalent group selected from the group consisting of an alkylene radical, a cycloaromatic radical, a heterocyclic radical, a carbonyl radical, an alkenylene radical, and combinations thereof;
wherein each of the alkyl radical, the alkenyl radical, the alkylene radical, the cycloaromatic radical, the heterocyclic radical, the carbonyl radical, and the alkenylene radical may be substituted with one or more groups selected from the group consisting of alkyl, hydroxyl, amino, monoalkylamino, dialkylamino, halogen, aryl, carboxyl, alkoxy, alkoxycarbonyl, hydrogenocarbonyl, sulphonato, amide and acyl groups.

37. The cosmetic composition according to claim 35, wherein the photodimerizable group is at least one monovalent radical selected from the group consisting of:
a stilbene; a styrylpyridinium (stilbazolium) of formula: 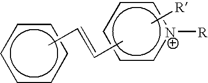
wherein
R represents a hydrogen atom, an alkyl group or hydroxyalkyl group, and
R′ represents a hydrogen atom or an alkyl group;
a styrylazolium of formula: 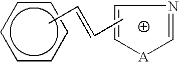
A denotes a sulphur atom, an oxygen atom, a group NR′ or a group C(R′)₂,
R represents a hydrogen atom, an alkyl group or hydroxyalkyl group, and
R′ represents a hydrogen atom or an alkyl group;
a styrylpyrazine; a chalcone; a (thio)cinnamate; a (thio)cinnamamide; a maleimide; a (thio)coumarin; a thymine; a uracil; a butadiene; an anthracene; a pyridine; a pyrrolizinone; an acridizinium salt; a furanone; a phenylbenzoxazole; and derivatives thereof.

38. The cosmetic composition according to claim 35, wherein the composition is free of photoinitiator.

39. The cosmetic composition according claim 35, wherein the photodimerizable group is grafted onto a support compound.

40. The cosmetic composition according to claim 39, wherein the support compound is at least one of a poly(vinyl) polymer and a polydiorganosiloxanes.

41. The cosmetic composition according to claim 40, wherein the support compound is a poly(vinyl acetate).

42. The cosmetic composition according to claim 40, wherein the support compound is a partially saponified poly(vinyl acetate).

43. The cosmetic composition according to claim 35, wherein the compound comprising at least one photodimerizable group is a polymer that is soluble or dispersed in the composition.

44. The cosmetic composition according to claim 35, wherein the compound comprising at least one photodimerizable group is adsorbed onto polymer particles which are dispersed in the composition.

45. The cosmetic composition according to claim 44, wherein the polymer particles are vinyl polymer particles.

46. The cosmetic composition according to claim 44, wherein the polymer particles are poly(vinyl acetate).

47. The cosmetic composition according to claim 44, wherein the composition is an aqueous dispersion of a partially saponified polyvinyl acetate polymer bearing stilbazolium groups, in contact or blended with polyvinyl acetate particles.

48. The cosmetic composition according to claim 35, wherein the compound comprising at least one photodimerizable group represents from 0.01% to 25% of the total weight of the composition.

49. The cosmetic composition according to claim 35, wherein the compound comprising at least one photodiamerizable group represents from 0.1% to 20% of the total weight of the composition.

50. The cosmetic composition according to claim 35, wherein the compound comprising at one photodimerizable group represents from 1% to 15% of the total weight of the composition.

51. The cosmetic composition according to claim 35, further comprising an effective amount of at least one photosensitizer.

52. The cosmetic composition according to claim 48, wherein the photosensitizer is at least one selected from the group consisting of thioxanthone, rose bengal, phloxin, eosin, erythrosin, fluorescein, acriflavine, thionine, riboflavine, proflavine, chlorophylls, haematoporphyrin, methylene blue, and mixtures thereof.

53. The cosmetic composition according to claim 51, wherein the photosensitizer is from 0.00001% to 5% of the total weight of the composition.

54. The cosmetic composition according to claim 35, wherein the cosmetic active agent is at least one of a hydrolysed or non-hydrolysed, modified or unmodified saccharide, oligosaccharide and polysaccharide; an amino acid; oligopeptide; a peptide; a hydrolysed or non-hydrolysed, modified or unmodified protein, polyamino acid and enzyme; a branched or unbranched fatty acid and fatty alcohol; an animal, plant or mineral wax; a ceramide and a pseudoceramide; a hydroxylated organic acid; a UV-screening agent; an antioxidant and free-radical scavenger; a chelating agent; an antidandruff agent; a seborrhoea regulator; a calmant; a cationic surfactant; a cationic or amphoteric polymer; an organomodified or non-organomodified silicone; a mineral, a plant or animal oil; a polyisobutene and poly(α-olefin); an ester; a soluble or dispersed anionic polymer; a soluble or dispersed nonionic polymer; a reducing agent; a dyestuff; a foaming agent; a film-forming agent; a particle; and mixtures thereof.

55. The cosmetic composition according to claim 54, wherein the cosmetic active agent is from 0.001% to 30% of the total weight of the composition.

56. The cosmetic composition according to claim 35, further comprising a cosmetically acceptable solvent or mixture of solvents.

57. The cosmetic composition according to claim 56, comprising at least one solvent selected from the group consisting of water, an alcohol, a polyol, a polyol ether, and mixtures thereof.

58. The cosmetic composition according to claim 56, comprising a C₁-C₆ lower alkanol.

59. The cosmetic composition according to claim 58, wherein the alkanol is at least one selected from the group consisting of ethanol, propanol and isopropanol.

60. The cosmetic composition according to claim 57, comprising at least one polyol selected from the group consisting of a propylene glycol, a hexylene glycol, a glycerol and a pentanediol.

61. The cosmetic composition according to claim 56, comprising a solvent in an amount of 0.1% to 99% of the total weight of the composition.

62. The cosmetic composition according to claim 35, having a pH of between 2 and 12.

63. The cosmetic composition according to claim 35, having a pH of between 3 and 11.

64. The cosmetic composition according to claim 35, further comprising at least one adjuvant selected from the group consisting of a fragrance, a filler, a pH regulator and a preserving agent.

65. The cosmetic composition according to claim 35, which is crosslinked.

66. The cosmetic composition according to claim 35, which is photo-crosslinked.

67. A hair styling composition comprising the cosmetic composition according to claim 35.

68. A process for treating keratin materials, comprising:
a) applying the composition of claim 35 to the keratin materials, and
b) irradiating the composition applied on the keratin materials to crosslink the composition.

69. The process according to claim 68, further comprising:
drying the keratin materials, before the irradiating and after the applying.

70. The process according to claim 68, further comprising:
before or after the irradiating, an acting time at room temperature or with heat, or under a red lamp.

71. The process according to claim 68, further comprising:
before the irradiating, partial masking of the keratin materials, and, after the irradiating, rinsing, or, when the keratin materials are hair, a shampooing or styling.

72. The process according to claim 68, further comprising:
before the partial masking of the keratin materials and after the applying, drying the keratin materials.

73. The process according to claim 68, wherein the irradiating is carried out with radiation in the UVA range or in the visible range.

74. The process according to claim 73, wherein the radiation is UVA radiation.

75. The process according to claim 68, wherein when the keratin materials are hair, and the process further comprises:
shaping the hair before or after the irradiating.

76. The process according to claim 68, wherein the compound comprising at least one photodimerizable group is heat-dimerizable, and the irradiating is heating between 40 and 200° C.

77. The process according to claim 68, wherein the keratin materials are hair or nails.
